(19) United States
(12) Patent Application Publication
(10) Pub. No.: US 2020/0038338 A1
(43) Pub. Date: Feb. 6, 2020

(54) LONG TERM DRUG DELIVERY BY TATTOO

(71) Applicants: Carlos Wambier, Hamden, CT (US); John Kulesza, Wethersfield, CT (US)

(72) Inventors: Carlos Wambier, Hamden, CT (US); John Kulesza, Wethersfield, CT (US)

(21) Appl. No.: 16/529,068

(22) Filed: Aug. 1, 2019

Related U.S. Application Data
(60) Provisional application No. 62/713,112, filed on Aug. 1, 2018.

(51) Int. Cl. A61K 9/70; A61K 9/00; A61M 37/00; A61K 45/06

(52) U.S. Cl. CPC A61K 9/7023; A61K 9/0014; A61M 37/0092; A61K 45/06; A61M 37/0084

(57) ABSTRACT

Provided are methods for long term drug delivery by tattoo. The bioactive agent, which comprises the drug, is delivered to a layer, or multiple layers, of the skin. Such a method allows for long-term drug activity the bioactive agent has a longer residence time within the skin when compared to routine delivery methods. Moreover, the bioactive agent may also be combined and mixed with a coloring composition, which signals the amount of bioactive agent that has been administered and if a reapplication of the bioactive agent is necessary. Also described are methods of utilizing such a drug delivery mechanism in order to effect cosmetic changes, such as enhancing hair growth by utilizing bioactive agents suited for enabling hair growth.

### CROSS REFERENCE TO RELATED APPLICATIONS

This application depends from and claims priority to U.S. Provisional Application No.: 62/713,112 filed Aug. 1, 2018, the entire contents of which are incorporated herein by reference.

### FIELD

The disclosure generally relates to bioactive agent delivery by tattooing. More specifically, the disclosure relates to methods for the administration of bioactive agents via a tattooing process.

### BACKGROUND

Delivery of bioactive agents via injection can be intradermal, subcutaneous, or intramuscular, among other. Subcutaneous delivery, most often used for skin level administration, typically involves the act of injecting a liquid comprising bioactive agents into a subcutaneous layer of skin using a hypodermic needle and a syringe. However, typical subcutaneous injections suffer from a variety of drawbacks including limited efficacy of certain drugs, relatively short half-lives of the bioactive agent(s) delivered, and ineffective adsorption of the bioactive agent(s) into the bloodstream. Further, the area treated by subcutaneous injections is highly localized if, for example, the bioactive agent is targeting a dermatological disorder. As such, individuals in need of subcutaneous injections are often required to make return visits to receive additional injections. This process can be both monetarily expensive and time intensive. Intradermal injections are difficult to properly achieve. As the dermis is extremely thin targeting this layer of the skin often results in injection being too deep or too shallow.

Intradermal injections can be very painful due to the presence of nerve termini in that area of the skin as well as the required high injection pressure due to the presence of dense collage fibers in this area of the skin. During and soon after a usual intradermal injection (a bolus), the liquid spreads to areas with less pressure, e.g. there is radial spread, deep spread to the subcutaneous tissue, and also to the lymphatic region as these areas present less resistance. Also, there is a possibility of inadvertently injecting in the lumen of a blood vessel (intraarterial or intravenous), which can cause embolism.

Medical tattoos have been used to ensure that instruments are properly located for repeated application of radiotherapy and to convey medical information about the wearer. Tattooing is a minimally invasive procedure that involves puncturing the skin with a vibrating needle in order to insert ink, dyes, pigments, or other microparticles into the epidermis or dermis layers of the skin. Typically, tattoos fall into three broad categories: purely decorative, symbolic, or pictorial. One such symbolic tattoo has historically included medical tattoos.

Early studies demonstrated using tattooing to deliver bioactive agents, such as DNA and molecular adjuvants, in order to investigate its effects on the human papillomavirus (HPV) type 16. It was found that DNA tattooing injections induce higher levels of specific antibodies and cellular immune responses than DNA-intramuscular injections. However, the method disclosed required four DNA tattooing injections over the course of 98 days, in order to achieve desirable results. As such, the regimen consisted of tattoo injections of DNA and other molecular adjuvants that have short residence times. As such, it was necessary to reapply the tattoo on multiple occasions in rapid succession. Such a repetitive tattooing regimen is both painful to the subject and time consuming.

A later study investigated the impact of injecting mice infected with cultured Leishmania (CL) infected macrophages with oleylphosphocholine (OlPC) via a tattooing process. While administering the OlPC liposomes by tattoo was shown to be more efficacious that topical ointments when treating the CL lesions, a number of downsides were realized. First, the mice were treated two times a day for five days, resulting in the administration of 10 total tattoos in order to achieve the desired results. As such, the tattooed skin sections all showed moderate to severe levels of focal ulceration with serocellular exudation and dermal fibrosis, which would likely be unacceptable if applied to a human subject. Similar to the problems described above, such an intensive tattooing regimen is highly painful, time consuming, and expensive.

One publication discloses techniques to treat androgenetic alopecia (hair loss) with minoxidil microinjections as an alternative to the topical application of minoxidil. (see Contin, L. A., Surgical and Cosmetic Dermatology, 2016; 8(2), 158-161). Subjects were subjected to minoxidil microinjections once a month for four months. No side effects were noted by the subjects, besides minor localized pain. However, there was no objective documentation of improvement.

Moreover, additional publications disclose delivering 5-fluorouracil to patches of skin showing signs of idiopathic guttate hypomelanosis (IGH) by tattooing process. (see Wambier, C. G. et al., Therapeutic pearl: 5-fluorouracil tattoo for idiopathic guttate hypomelanosis, Journal of the American Academy of Dermatology, 2018; 78(4), e81-e82; Arbache, S., Activation of melanocytes in idiopathic guttate hypomelanosis after 5-fluorouracil infusion using a tattoo machine: Preliminary analysis of a randomized, split-body, single blinded, placebo controlled clinical trial. Journal of the American Academy of Dermatology, 2018; 78(1), 212-215). These references note that the initial tests indicate that 5-fluorouracil may be beneficial in treating IGH, but that additional studies must be conducted in order to show the safety and efficacy of delivering 5-fluorouracil by a tattooing process.

U.S. Pat. No. 9,072,678 discloses a method that involves localized delivery of low dosages of bioactive compounds via biodegradable tattoos. While the bioactive compounds are described, in some embodiments, as “sustained release” or “long term release,” the disclosure makes it clear that long-term release mechanisms are still “unknown” and “yet to be discovered.” Moreover, there is no mention in the reference disclosing the cosmetic benefits, such as administering bioactive agents in order to promote hair growth or other permanent cosmetic alterations.

As the prior studies demonstrate, drug delivery by tattoo systems suffer from the quick dissipation or degradation of the drug itself after it has been delivered. Such dissipation or degradation leads to the necessity of re-applying the tattoo, which is painful, time consuming, and expensive.

As such, new materials and methods are needed in order to more effectively target bioactive agents to a desired site within a desired layer of the skin and improve the residence time of drugs, such as medicated substances, delivered by tattoo.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Provided in this disclosure are materials and methods for delivering bioactive agents, such as medicinal substances, to the one or more layers of skin by a tattooing process. It was found that certain bioactive agent formulations lead to a longer drug residence time within the skin. By increasing the residence time of the bioactive agent (e.g. combining with a polymeric moiety or in large or other salt form) or using a bioactive agent with a suitably long residence time, the formulations and methods of delivery improve the efficacy of the bioactive agent. As such, the method for delivering bioactive agents, such as medicated substances, includes delivering a bioactive agent into one or more layers of the skin, optionally the dermis, by a tattooing process. In some aspects, the process further includes delivering bioactive agents by a tattooing process that stimulates hair growth in order to treat or reduce baldness.

### DETAILED DESCRIPTION

This disclosure presents the unexpected result that long term delivery of drugs by tattoo formulated for extended release in the skin allows for improved acceptability. Such long term delivery of bioactive agents may span days, weeks, months, years, or even decades depending on the residence time of the bioactive agent within the skin. Administration of one or more bioactive agents is performed by providing an oscillating needle that is fluidly connected to a composition comprising the bioactive agent(s). The bioactive agent is then administered with the oscillating needle into a targeted layer of skin. The layer of skin may be dermal, epidermal, or subcutaneous tissue, and the residence time of the bioactive agent is at least 24 hours.

As used herein, “bioactive” can mean: one or all of the activities of a compound that show pharmacological or biological activity in human or animal body. Such biological activity is preferred to have a therapeutic or cosmetic effect. Substances or compounds that are bioactive are referred to as “drugs,” “therapeutic agents,” “bioactive agents,” “bioactive compounds,” or “medicated substances.”

As used herein, the terms “subject” or “organism” are treated synonymously and are defined as any organism with skin. A subject illustratively includes a mammal, humans, non-human primates, horses, goats, cows, sheep, pigs, dogs, cats, rodents, birds, fish, etc.

As used herein the term “tattoo” does not require coloring of the skin, but is a process similar to the decorative tattooing process wherein color or design is imparted to the skin. Similar devices may be used for decorative tattooing as are used in the present disclosure as will be described herein below. Tattoo as used herein is the process of delivery of one or more bioactive agents to a desired layer of the skin, optionally the dermis, where the bioactive agent may be administered alone, as part of a solution or suspension, and/or with a polymeric moiety or in other form (e.g. salt) so as to increase the residence time of the bioactive agent in the skin.

Administration by tattoo differs from epidermal delivery as it is previously known. For example, epidermal delivery or other prior delivery mechanisms involve making punctures over the surface of the skin and then spreading or rubbing a topical formulation on the perforated skin. The punctures will have no negative pressure. Quite the contrary, body fluids expelled by the punctures exhibit a pressure higher than atmospheric pressure. As such, by these prior processes only topical medications could permeate the holes. Tattooing as used herein is a form of direct dermal delivery (in some aspects). A solid (e.g. sterile metal needle) perforates through the epidermis to the dermis, and is removed at high speed. This removal creates a negative pressure formed in the space created by the needle. This pressure sucks down any fluid located over the hole just formed. This means that if a liquid layer (e.g. droplet, present on or within a needle, etc.) is present above the micro puncture, the liquid and any material carried in the liquid (e.g. bioactive agent/polymeric moiety) will be sucked into the hole and delivered efficiently to the desired skin layer, illustratively the dermis. This mechanism also avoids inadvertent intravascular injections because the pressure is stabilized once the fluid reaches the dermis.

The processes as provided herein administer one or more bioactive agents to a targeted layer of the skin, optionally the dermis, optionally the subcutaneous tissue, optionally the epidermal layer, depending on the depth of the puncture of the needle by the tattoo machine. The bioactive agents that may be used include, but are not limited to: hair growth agents, optionally minoxidil; antiviral agents; anti-infectives; antibiotics; antiviral agents; antifungal agents; antibacterial agents; antipruritics; anticancer agents, antipsychotics; cholesterol- or lipid-reducing agents; cell cycle inhibitors; anticancer agents; anti-parkinsonism drugs; HMG-CoA inhibitors; antirestenosis agents; anti-inflammatory agents; antiasthmatic agents; anthelmintic; immunosuppressives; muscle relaxants; antidiuretic agents; vasodilators; nitric oxide; nitric oxide-releasing compounds; beta-blockers; hormones; antidepressants; decongestants; calcium channel blockers; bone growth factors; bone morphogenic proteins; wound healing agents; analgesics; local anesthetic agents; antihistamines; sedatives; angiogenesis-promoting agents; angiogenesis-inhibiting agents; tranquilizers; sunscreen compositions; radiation blocking material; ultraviolet protection agents; chemotherapeutical agents; and the like, which can be therapeutic as well as toxins. In some aspects, a bioactive agent is an antibody. In some aspects, a bioactive agent is a cell. Illustrative examples of cells that can be used for therapeutic use in the processes as provided herein, may include, but are not limited to mammalian cells including stem cells; cellular components or fragments, such as enzymes, DNA, RNA, and genes may also be included as bioactive components or drugs.

As such, the processes as provided herein may be used to treat a disease of condition of a subject. A disease of condition for which the process may be used to treat, prevent, or prevent the continuance of is illustratively any skin condition. Illustrative examples of a skin condition include but are not limited to acanthosis nigricans, acne, acne scars, actinic keratosis, alopecia areata, atopic dermatitis, basal cell carcinoma, baldness, bed bugs, birthmarks, botulinum toxin, cellulitis, cold sores, contact dermatitis, dandruff, dermatofibrosarcoma protuberans, dyshidrotic eczema, eczema, hair loss, herpes simplex, hidradenitis suppurativa, hives, hyperhidrosis, impetigo, ichthyosis vulgaris, keloids, keratosis pilaris, lichen planus, melanoma, melisma, merkel cell carcinoma, moles, molluscum contagiosum, neurodermatitis, nummular dermatitis, pemphigus, pityriasis rosea, psoriasis, psoriatic arthritis, ringworm, rosacea, scabies, scalp psoriasis, scars, scerloderma, sebaceous carcinoma, seborrheic dermatitis, seborrheic keratosis, shingles, skin cancer, squamous cell carcinoma, stasis dermatitis, tinea versicolor, vitiligo, and warts.

A bioactive agent is one that is known to or is newly developed to treat, prevent, or prevent the continuance of a disease or condition, not limited to diseases or conditions of the skin. In some aspects of the disclosure, suitable bioactive agents may be small-molecule (e.g. less than or equal to 1000 Da, optionally less than or equal to 500 Da) drugs. Suitable types of small-molecule drugs may include, but are not limited to: triamcinolone; triamcinolone acetonide; methylprednisolone; betamethasone; betamethasone acetate; sodium phosphate; dexamethasone sodium phosphate; corticosteroids, such as hydrocortisone sodium succinate, dexamethasone sodium phosphate, cortisone, hydrocortisone, cortisone acetate, prednisone, cortodoxone, desoxycortone, and the like; imiquimod; minoxidil; alfatradiol; dutasteride; finasteride; bicalutamide; cyproterone acetate; flutamide; spironolactone; topilutamide; amifampridine; nepidermin; janus kinase inhibitors, such as tofacitinib, ruxolitinib, baricitinib, PD4 inhibitors, calcineurin inhibitors, prostaglandin analogues, and the like; or any derivatives or combinations thereof.

In some aspects of the disclosure, the residence time of the bioactive agent delivered via tattoo may be at least 24 hours, 48 hours, one week, one month, 6 months, one year, two years, five years, or longer. In other aspects, the residence time of the bioactive agent may be from one day to two days, one day to one week, one week to two weeks, two weeks to one month, one week to one month, one month to two months, two months to six months, one month to six months, six months to one year, two months to one year, one month to one year, one year to two years, one year to three years, one year to four years, one year to five years, five years to ten years, one year to ten years, or decades from the time of tattoo application. It is appreciated that such delivery of a bioactive agent may persist throughout the target's lifetime. Residence time is used herein as the time a bioactive agent can be detected or has biological or pharmacological activity at the site of injection.

Such increased drug residence time may be achieved by combining the bioactive agent with one or more polymeric moieties or other moieties that inhibit absorption of the drug to the system of a subject. A bioactive agent is optionally combined in a suspension of polymeric moiety, in a solution of polymeric moiety, is entrapped within a polymeric moiety, or is covalently or non-covalently bound to a polymeric moiety. Optionally, 1, 2, 3, 4, or more of the same or differing polymeric moieties may be used.

Illustrative examples of polymeric moieties are those with a molecular weight ranging from 200 to 100,000 Daltons. Polymeric moieties are optionally linear, branched, liable, or combinations thereof. The polymeric moieties are optionally homomeric or heteromeric. Illustrative examples of polymeric moieties include one or more molecules of carbohydrate, polyprepolymer, or polyoxyethylene (otherwise known as polyethylene glycol or “PEG”).

Illustrative examples of polymeric moieties include but are not limited to: polyalkyl alcohols and glycols (including heteroalkyl with, for example, oxygen) such as polyoxyethylenes and polyoxyethylene derivatives; dextrans including functionalized dextrans; styrene polymers; polyethylene and derivatives; polyanions including, but not limited to polymers of heparin, polygalacturonic acid, mucin, nucleic acids and their analogs including those with modified ribosephosphate backbones, polypeptides of glutamate, aspartate, or combinations thereof, as well as carboxylic acid, phosphoric acid, and sulfonic acid derivatives of synthetic polymers; and polycations, including but not limited to, synthetic polycations based on acrylamide and 2-acrylamido-2 methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and proteins such as protamine, the histone polypeptides, polylysine, polyarginine and polyornithine, and mixtures and derivatives thereof.

Optionally, a PEG based copolymer such as PEG-polylactate-10 that dissolves in water is used for delivering the bioactive agent by tattoo. The polymer forms micelles in water and thus the emulsion in water can dissolve/emulsify hydrophobic small molecular weighted drugs and can release the drug in a sustained manner. Some compositions may undergo swelling (absorption of water) after the solvent has been extracted. The amount of water absorption will depend upon the amount of PEG in the copolymer. In general, as percentage of PEG in the copolymer is increased, the copolymer will absorb more water. The amount of water absorbed by in situ formed particle may range from 0.1 percent up to 10000 percent, generally 0.5 to 200 percent, depending on hydrophilic nature of the polymer used.

Polyoxyethylene is optionally end capped where one end is end-capped with a relatively inactive group such as an alkoxy group, while the other end is a hydroxyl group that may be further modified by linker moieties. When the term “PEG” is used to describe polyoxyethylene the term “PEG” may be followed by a number (not being a subscript) that indicates a PEG moiety with the approximate molecular weight equal the number. Hence, “PEG10000” is a PEG moiety having an approximate molecular weight of 10,000 Daltons. PEG400 has an approximate molecular weight of 400 Daltons (e.g. 380-420 g/mol).

In some aspects, a polymeric moiety is covalently bound to a bioactive agent. The term “PEGylation” as used herein denotes modification of a bioactive agent by attachment of one or more PEG moieties, optionally via a linker, at one or more sites in a bioactive molecule. The PEG moiety is illustratively attached by nucleophilic substitution (acylation) on N-terminal a-amino groups or on lysine residue(s) on the gamma-positions, e.g., with PEG-succinimidyl esters. Optionally, polyoxyethylene moieties are attached by reductive alkylation—also on amino groups present in the bioactive agent using PEG-aldehyde reagents and a reducing agent, such as sodium cyanoborohydride. Optionally, polyoxyethylene moieties are attached to the side chain of an unpaired cysteine residue in a Michael addition reaction using for example PEG maleimide reagents.

Optionally a polymeric moiety is a polyprepolymer, optionally a PPG-12/SMDI copolymer. An illustrative example of a polyprepolymer is HO(C₃H₆O)₁₂[C₁₅H₂₂N₂O₂(C₃H₆O₁₂]ₘH where m is 1-4.

In some aspects of the disclosure, the injection of the bioactive agent via tattoo comprises low dosages. Low dosage is defined as less than about 2.0 milliliters (mL) per injection, or alternatively from about 1.0×10⁻²⁰ mL to about 2 mL, from about 1.0×10⁻²⁰ mL to about 1 mL, from about 1.0×10⁻²⁰ mL to about 0.5 mL, from about 1.0×10⁻²⁰ mL to about 0.3 mL, from about 1.0×10⁻²⁰ mL to about 0.2 mL, from about 1.0×10⁻²⁰ mL to about 0.1 mL, from about 1.0×10⁻²⁰ mL to about 1.0×10⁻² mL, from about 1.0×10⁻²⁰ mL to about 1.0×10⁻³ mL, from about 1.0×10⁻²⁰ mL to about 1.0×10⁻⁵ mL, from about 1.0×10⁻²⁰ mL to about 1.0×10⁻¹⁰ mL, from about 1.0×10⁻²⁰ mL to about 1.0×10⁻¹⁵ mL, from about 1.0×10⁻¹⁵ mL to about 2 mL, from about 1.0×10⁻¹⁵ mL to about 1 mL, from about 1.0×10⁻¹⁵ mL to about 0.5 mL, from about 1.0×10⁻¹⁵ mL to about 0.3 mL, from about 1.0×10⁻¹⁵ mL to about 0.2 mL, from about 1.0×10⁻¹⁵ mL to about 0.1 mL, from about 1.0×10⁻¹⁵ mL to about 1.0×10⁻² mL, from about 1.0×10⁻¹⁵ mL to about 1.0×10⁻³ mL, from about 1.0×10⁻¹⁵ mL to about 1.0×10⁻⁵ mL, from about 1.0×10⁻¹⁵ mL to about 1.0×10⁻¹⁰ mL, from about 1.0×10⁻¹⁰ mL to about 2 mL, from about 1.0×10⁻¹⁰ mL to about 1 mL, from about 1.0×10⁻¹⁰ mL to about 0.5 mL, from about 1.0×10⁻¹⁰ mL to about 0.3 mL, from about 1.0×10⁻¹⁰ mL to about 0.2 mL, from about 1.0×10⁻¹⁰ mL to about 0.1 mL, from about 1.0×10⁻¹⁰ mL to about 1.0×10⁻² mL, from about 1.0×10⁻¹⁰ mL to about 1.0×10⁻³ mL, from about 1.0×10⁻¹⁰ mL to about 1.0×10⁻⁵ mL, from about 1.0×10⁻⁵ mL to about 2 mL, from about 1.0×10⁻⁵ mL to about 1 mL, from about 1.0×10⁻⁵ mL to about 0.5 mL, from about 1.0×10⁻⁵ mL to about 0.3 mL, from about 1.0×10⁻⁵ mL to about 0.2 mL, from about 1.0×10⁻⁵ mL to about 0.1 mL, from about 1.0×10⁻⁵mL to about 1.0×10⁻² mL, from about 1.0×10⁻⁵ mL to about 1.0×10⁻³ mL, or any other suitable range from about 1.0×10⁻²⁰ mL to about 2 mL per injection.

In additional aspects of the disclosure, the pH of the bioactive agent composition (liquid in which the bioactive agent is present for administration) administered to the subject ranges from about two to about ten, or alternatively from about two to about nine, from about two to about eight, from about two to about seven, from about two to about six, from about two to about five, from about two to about four, from about three to about eight, from about three to about seven, from about three to about six, from about three to about five, or any other suitable range from about two to about twelve. In a one illustrative aspect of the disclosure, the pH of the bioactive agent ranges from about three to about eight.

The bioactive agent may be administered at any desirable concentration which will be selectable by one of ordinary skill in the art depending on the solubility or desired compositional aspects of the bioactive agent composition, the intended residence time, toxicity, efficacy, etc. Illustratively, a bioactive agent is administered at 0.001 weight percent to 10 weight percent, or any value or range therebetween. One advantage of the processes as used herein is that the combination of the bioactive agent with the polymeric moiety may allow delivery of concentrations of the bioactive agent not achievable by other methods. As such, a bioactive agent is optionally administered at a concentration above the solubility limit of the bioactive agent in water. Optionally, the bioactive agent is administered at a concentration that is above that used for oral delivery, subcutaneous delivery, injection, or other delivery method.

The bioactive agent may also be mixed with coloring compositions. Such a mixture allows the amount of bioactive agent that has been injected via tattooing to be visualized. Not only would such a mixture act as a visual aid so that the tattoo applier can see how much of the bioactive agent has been applied, but would also allow the tattoo recipient the option of choosing his or her own tattoo design, procedure permitting. Examples of “coloring compositions” include any coloring composition or chemical that is suitable for human or animal implantation and are preferably approved by the Food and Drug Administration (FDA) for use in implantable medical devices. In some aspects, coloring compositions that are biodegradable may be used. The fading of such biodegradable coloring compositions may align with the residence time of the bioactive agent. This would allow the tattoo wearer and/or the tattoo applier to gauge whether an additional injection is necessary.

The coloring compositions may include, but are not limited to heavy metal ink mixtures such as, but not limited to, mixtures comprising mercury, lead, cadmium, nickel, zinc, chromium, cobalt, aluminum, titanium, copper, iron, barium, and combinations thereof. Metal oxides, azo-chemicals, naptha-derived chemicals, carbon, and other natural pigments are also suitable as components of an ink mixture. The coloring compositions may also include, but are not limited to: methylene blue; eosin Y; fluorescein sodium; chromium-cobalt-aluminum oxide; ferric ammonium citrate; pyrogallol; logwood extract; 1,4-bis(2-hydroxyethyl)aminol-9,10-anthracenedione; bis(2-propenoic)ester copolymers; 1,4-bis((2-methylphenyl)amino)-9,10-anthracenedione; 1,4-bis(4-(2-methacryloxyethyl)phenylamino)anthraquinone copolymers; carbazole violet; chlorophyllin-copper complex, oil soluble; chromium-cobalt aluminum oxide; chromium oxide greens; C.I. Vat Orange 1; 2-((2,5-Diethoxy-4-((4-methylphenyl)thiol)phenyl)azo-1,3,5-benzenetriol; 1 6,23 -Dihydrodinaphtho(2,3-a:2′,3′-i)naphtha(2′,′:6,7)indolo(2,3-c)carbazole-5,10,15,17,22,24-hexone; N,N′-(9,10-Dihydro-9,10-dioxo-1,5-anthracenediyl)bis benzamide; 7,16-Dichloro-6,15-dihydro 5,9,14,18-anthrazinetetrone; 16,17-Dimethoxydinaphtho(1,2,3-cd:3′,2′, 1′-lm)perylene-5,10-dione; Poly(hydroxyethyl methacrylate)-dye copolymers: one or more of Reactive Black 5; Reactive Blue 21; Reactive Orange 78; Reactive Yellow 15; Reactive Blue No. 19; Reactive Blue No. 4; C.I. Reactive Red 11; C.I. Reactive Yellow 86; C.I. Reactive Blue 163; C.I. Reactive Red 180: 4-[(2,4-dimethylphenyl)azo]-2,4-dihydro-5-methyl-2-phenyl-3H-pyrazol-3-one; 6-ethoxy-2-(6-ethoxy-3-oxobenzo[b]thien-2(3H)-ylidene)benzo [b] thiophen-3(2H)-one; phthalocyanine green; iron oxides; titanium dioxide; vinyl alcohol/methyl methacrylate-dye reaction products; one or more of: (1) C.I. Reactive Red 180; C.I. Reactive Black 5: C.I. Reactive Orange 78; C.I. Reactive Yellow 15: C.I. Reactive Blue 19: C.I. Reactive Blue 21; mica-based pearlescent pigments; C.I. Reactive Blue 69; D&C Blue No. 9; D&C Green No. 5; [phthalocyaninato(2-)]copper; FD&C Blue No. 2; D&C Blue No. 6; D&C Green No. 6; D&C Red No. 17; D&C Violet No. 2; D&C Yellow No. 10; and combinations thereof.

In some aspects of the disclosure, the bioactive agent may be applied by tattoo to any layer of skin as found at understood depths from the outer surface of the skin, including the epidermis, dermis, subcutaneous layer, or combinations thereof. Such depths may be from about 0.01 mm to about 10 mm, about 0.02 mm to about 10 mm, about 0.05 mm to about 10 mm, 0.1 mm to about 10 mm, about 0.2 mm to about 10 mm, about 0.5 mm to about 10 mm, about 1 mm to about 10 mm, about 2 mm to about 10 mm, about 5 mm to about 10 mm, about 0.1 mm to about 5 mm, about 0.2 mm to about 5 mm, about 0.5 mm to about 5 mm, about 1 mm to about 5 mm, about 2 mm to about 5 mm, about 0.01 mm to about 2 mm, about 0.02 mm to about 2 mm, about 0.05 mm to about 5 mm, about 0.1 mm to about 2 mm, about 0.2 mm to about 2 mm, about 0.5 mm to about 5 mm, about 1 mm to about 5 mm, about 2 mm to about 5 mm, about 0.01 mm to about 2 mm, about 0.02 mm to about 2 mm, about 0.05 mm to about 2 mm, about 0.1 mm to about 2 mm, about 0.2 mm to about 2 mm, about 0.5 mm to about 2 mm, about 1 mm to about 2 mm, about 0.01 mm to about 1 mm, about 0.02 mm to about 1 mm, about 0.05 mm to about 1 mm, about 0.1 mm to about 1 mm, about 0.2 mm to about 1 mm, about 0.5 mm to about 1 mm, about 0.01 mm to about 0.5 mm, about 0.02 mm to about 0.5 mm, about 0.01 mm to about 0.2 mm, about 0.01 mm to about 0.5 mm, about 0.2 mm to about 0.5 mm, about 0.1 mm to about 0.2 mm, or any other range between 0.01 mm to about 10 mm.

In certain aspects of the disclosure, the tattoo needle used to apply the bioactive agent may be any commercially-available tattoo needle. Suitable types of tattoo needles include, but are not limited to, round shaders, round liners, super tight round liners, extra super tight round liners, flats, magnums, curved magnums, or stacked magnums. Suitable diameters for the needle range from about 0.15 mm to about 0.50 mm in diameter. For example, tattoo needles #6, #8, #10, #12, and #13 all range from 0.20 mm to 0.40 mm in diameter and are considered suitable. The number of tattoo needle points in a grouping may range from one to 100 needles. In one preferred embodiment, the grouping comprises a plurality of needle points numbering between three and 15 needle points. The taper point length of the needle may range from about 1.5 mm to about 8 mm. Such suitable taper point lengths include the standard taper, long taper, double long taper, extra long taper, super long taper, and extra super long taper needles. Any combination of needle type, diameter, point, and taper length described above is considered suitable for purposes of applying a bioactive agent via tattoo.

Moreover, the tattooing device, colloquially referred to as a tattoo gun, used to apply the bioactive agent may be any commercially-available tattoo gun. Suitable types of tattoo guns may include, but are not limited to, rotary, coil, liner, shader, or pneumatic tattoo machines. The tattoo gun may operate at a frequency of from about 20 hertz to about 150 hertz, about 40 hertz to about 130 hertz, about 60 hertz to about 110 hertz, or any other suitable range between about 20 hertz to about 150 hertz.

Various aspects of the present invention are illustrated by the following non-limiting examples. The examples are for illustrative purposes and are not a limitation on any practice of the present invention. It will be understood that variations and modifications can be made without departing from the spirit and scope of the invention.

### EXAMPLES

### Example 1

### Treatment of Alopecia

A suspension of minoxidil suspension at 5 wt % is created in a PEG 400 vehicle. As a control minoxidil is added to water to saturation. The test and control agents are delivered by tattoo machine to the dermis on independent sides of the face (either beard, eyebrow, or scalp) of the same subject once every 2 months for 6 months (i.e. 3 administrations). Three different subjects were tested. The side that was treated in the beard or scalp had approximately 300% more terminal (thick, strong) hairs per unit area, after 6 months (3 sessions). This compares to a comparable required administration of minoxidil solution per prior uses (every 2 weeks or every 4 weeks).

### Example 2

### Treatment of Vitiligo

To evaluate the ability of the process to treat vitiligo in a subject suffering the condition, one or more Janus kinase inhibitors may be administered by the tattooing process. A suspension of tofacitinib may be made in PEG such as PEG 400 or other suitable polymer. Alternatively, PEG may be covalently bound to tofacitinib via a desired linker. The resulting bioactive agent composition may then be administered at a 2 weight percent (wt %) concentration of the tofacitinib to a desired area of the skin of the subject direct to the dermal layer by a tattoo machine. It is expected that pigmentation in 90% of patches will be resolved in the first 6 months. As a placebo control, this may be compared to a second region of skin with NaCl0.9 wt % followed by tofacitinib cream 2 wt % twice a day for the same period. As is expected by control only 25% regimentation is observed.

### Example 3

### Treatment of Psoriasis

A composition of methotrexate at 25 mg/ml is prepared in polyethylene glycol. This is administered to subjects at the site of psoriatic plaques by tattoo machine directly to the dermal layer. Administration will be once every 3 months for 9 months. As a control, subjects will be administered 25 mg/ml methotrexate by injection once a month for the same period. Subjects undergoing the tattoo administration of the increased residence time methotrexate will show clearance following the first treatment session. Control subjects will suffer traditional side effects such as increased liver enzymes and show similar levels of clearance as the tattoo administration.

### Example 4

### Treatment of Basal Cell Carcinoma (BCC)

A composition of vismodegib is prepared in suspension with polyethylene glycol. This is administered to subjects at the site of carcinoma by tattoo machine. Administration will be once a week for 6 months. As a control, subjects will be administered vismodegib orally for the same period. Subjects undergoing the tattoo administration of the increased residence time vismodegib will show clearance of 90% of the BCC. Control subjects will suffer traditional side effects such as muscle tremors, loss of appetite, reduction of smell and taste, and only 50% of the BCCs will resolved in the first 6 months.

The foregoing description of particular aspect(s) is merely exemplary in nature and is in no way intended to limit the scope of the disclosure, its application, or uses, which may, of course, vary. The materials and processes are described with relation to the non-limiting definitions and terminology included herein. These definitions and terminology are not designed to function as a limitation on the scope or practice of the disclosure, but are presented for illustrative and descriptive purposes only. While the processes or compositions are described as an order of individual steps or using specific materials, it is appreciated that steps or materials may be interchangeable such that the description of the disclosure may include multiple parts or steps arranged in many ways as is readily appreciated by one of skill in the art.

It will be understood that, although the terms “first,” “second,” “third,” etc. may be used herein to describe various elements, components, regions, and/or layers, these elements, components, regions, and/or layers should not be limited by these terms. These terms are only used to distinguish one element, component, region, or layer from another element, component, region, or layer. Thus, “a first ‘element,’” “component,” “region,” or “layer,” discussed below could be termed a second (or other) element, component, region, or layer without departing from the teachings herein.

The terminology used herein is for the purpose of describing particular aspects of the disclosure only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Various modifications of the present disclosure, in addition to those shown and described herein, will be apparent to those skilled in the art of the above description. Such modifications are also intended to fall within the scope of the appended claims.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular aspects of the disclosure, but is not meant to be a limitation upon the practice thereof.

1. A method for delivering a bioactive agent, the method comprising:
providing an oscillating needle that is fluidly connected to a composition comprising a bioactive agent; and
administering the bioactive agent with the oscillating needle into a layer of skin,
wherein the layer of skin is dermal, epidermal, or subcutaneous tissue; and
the bioactive agent has a residence time of at least 24 hours in the layer of skin.

2. The method of claim 1, wherein the bioactive agent comprises one or more of: hair growth agents; antiviral agents; anti-infectives; antibiotics; antiviral agents; antifungal agents; antibacterial agents; antipruritics; anticancer agents, antipsychotics; cholesterol- or lipid-reducing agents; cell cycle inhibitors; anticancer agents; anti-parkinsonism drugs; HMG-CoA inhibitors; antirestenosis agents; anti-inflammatory agents; antiasthmatic agents; anthelmintic; immunosuppressives; muscle relaxants; antidiuretic agents; vasodilators; nitric oxide; nitric oxide-releasing compounds; beta-blockers; hormones; antidepressants; decongestants; calcium channel blockers; bone growth factors; bone morphogenic proteins; wound healing agents; analgesics; local anesthetic agents; antihistamines; sedatives; angiogenesis-promoting agents; angiogenesis-inhibiting agents; tranquilizers; sunscreen compositions; radiation blocking material; ultraviolet protection agents; chemotherapeutical agents, or combinations thereof.

3. The method of claim 2, wherein the bioactive agent is triamcinolone, triamcinolone acetonide, methylprednisolone, betamethasone, betamethasone acetate, sodium phosphate, dexamethasone sodium phosphate, hydrocortisone sodium succinate, dexamethasone sodium phosphate, cortisone, hydrocortisone, cortisone acetate, prednisone, cortodoxone, desoxycortone, imiquimod, minoxidil, alfatradiol, dutasteride, finasteride, bicalutamide, cyproterone acetate, flutamide, spironolactone, topilutamide, amifampridine, nepidermin, tofacitinib, ruxolitinib, baricitinib, PD4 inhibitors, calcineurin inhibitors, prostaglandin analogues, or any derivatives or combinations thereof.

4. The method of claim 1, wherein the dosage of the bioactive agent delivered ranges from about 1.0×10⁻²⁰ mL to about 2 mL per injection.

5. The method of claim 1, wherein the pH of the bioactive agent composition delivered ranges from about two to about ten.

6. The method of claim 1, wherein the bioactive agent is mixed with a coloring composition.

7. The method of claim 6, wherein the coloring composition is selected from the group consisting of heavy metal ink mixtures, metal oxide mixtures, azo-chemical mixtures, naptha-derived chemical mixtures, carbon, and mixtures comprising other natural pigments.

8. The method of claim 1, wherein the bioactive agent has a residence time of at least 48 hours in the layer of skin.

9. The method of claim 1, wherein the bioactive agent is applied to skin at depths of from about 0.1 mm to about 10 mm.

10. The method of claim 1, wherein the oscillating needle is a tattoo needle.

11. The method of claim 10, wherein the tattoo needle is selected from the group consisting of round shaders, round liners, super tight round liners, extra super tight round liners, flats, magnums, curved magnums, or stacked magnums.

12. The method of claim 10, wherein the tattoo needle comprises a diameter of from about 0.15 mm to about 0.50 mm.

13. The method of claim 10, wherein the tattoo needle comprises at least one tattoo needle point.

14. The method of claim 13, wherein the number of tattoo needle points ranges from one to 100, or from 3 to 15 needle points.

15. The method of claim 10, wherein the needle comprises a taper point length of from about 1.5 mm to about 8 mm.
